(19) United States
(12) Patent Application Publication
(10) Pub. No.: US 2007/0229759 A1
(43) Pub. Date: Oct. 4, 2007

(54) Sunglass retention device for headgear

(76) Inventor: Robert B. Jones, Arlington, GA (US)

Correspondence Address: JAMES RAY & ASSOCIATES, 2640 Pitcairn Road, Monroeville, PA, 15146, US

(21) Appl. No.: 11/724,471

(22) Filed: Mar. 15, 2007

Related U.S. Application Data
(60) Provisional application No. 60/782,405, filed on Mar. 15, 2006.

(51) Int. Cl. A61B 3/00

(52) U.S. Cl. 351/200

(57) ABSTRACT

A sunglasses-retention kit to enable attachment of sunglasses to headgear includes at least one substantially flexible hook and loop (Velcro) strip engageable to the headgear and having each of a predetermined length and a predetermined width, wherein the Velcro strip includes hooks and/or loops over at least one-third of the strip; and, at least one adhesive-backed hook and loop pad engageable to the sunglasses for mating to the flexible hook and loop strip.

### CROSS REFERENCE TO RELATED APPLICATION

This patent application is related to and claims priority from U.S. Provisional Patent Application Ser. No. 60/782,405 filed Mar. 15, 2006.

### FIELD OF THE INVENTION

The present invention relates, in general, to headgear and, more particularly, this invention relates to an apparatus for holding sunglasses to the top side of a ball cap, ski hat, or visor during periods when the glasses are not being worn to shield ones eyes.

### BACKGROUND OF THE INVENTION

Prior to the conception and development of the present invention, people working or playing outdoors in sunny weather would frequently wear a cap or visor along with sunglasses to protect both their eyes and their faces. Typical examples would be those golfing, skiing or playing tennis, softball and baseball. During periods when a cloud temporarily covers the sun, or when the wearer goes indoors, the sunglasses are removed to enable better vision, but then there is a problem of where to put the sunglasses. The wearer wants to have them readily available when suddenly needed again, and without having to go back to a golf cart or return to the bench.

Prior art provides numerous examples of headgear with a built-in means to hold eyeglasses when not needed in front of the eyes, beginning with U.S. Pat. No. 264,574 which illustrates a means for holding spectacles underneath the brim of a hat. U.S. Pat. No. 857,838 teaches a pivot arm mechanism attached to the sides of a cap and connecting to the temples of eyeglasses or goggles. U.S. Pat. No. 4,179,753 shows means for holding sunglasses to several types of headgear by attaching a clip in the front above the bill and small built-in pockets along the sides for retaining the ends of the temples.

U.S. Pat. No. 5,860,167 illustrates how many items, including sunglasses, could be held to a ball cap. U.S. Pat. No. 6,237,159 illustrates how dual parallel button holes on both sides of a ball cap could be used to hold sunglasses above the bill. U.S. Pat. No. 6,647,554 shows a now commercially available cap with sewn-on multi-position retention patches. U.S. Pat. No. 6,185,748 teaches an elastic fabric band sewn onto the top surface of the bill of a cap for retention of a folded pair of eye glasses.

One common feature of all the aforementioned prior art devices is that the retention means are built in as part of the headgear. This means that the customer must purchase that particular cap, something not normally desirable, or practical in the case of baseball or softball teams that all wear identical ball caps for one season. An exception to this is provided by Morris in U.S. Pat. No. 6,481,059 which teaches a clip-on design. However, a drawback with this design and many of those referenced above it that it is likely to require removing the hat or considerable fumbling around to engage the sunglasses with the retention means. The inside portion of the clip could also be annoying to the wearer. U.S. Pat. No. 4,768,231 teaches the use of Velcro tabs to secure sunglasses while being worn in front of the users eyes, but this design also requires an undesirable amount of time to engage or disengage each temple separately. Morris attempted to address these issues in U.S. Pat. No. 6,668,426 with an after-market attachment having a broad Y-shaped insertion end. This design would work well with straight-back temples, but not well with many sunglasses which have temples curved at the ends to partially wrap around the ears.

Hence, there is still a need for a system to temporarily retain common existing sunglasses on existing hats with a quick on-and-off feature.

### SUMMARY OF THE INVENTION

The present invention provides a means to turn virtually any headgear into one that will conveniently retain ordinary sunglasses close at hand during periods when the sunglasses are not being worn. According to a first aspect of the invention, described is a sunglasses-retention kit to enable attachment of sunglasses to headgear and it includes at least one substantially flexible hook and loop (Velcro) strip engageable to the headgear and having each of a predetermined length and a predetermined width, wherein the Velcro strip includes hooks and/or loops over at least one-third of the strip; and, at least one adhesive-backed hook and loop pad for mating to the flexible hook and loop strip.

In a second aspect, an apparatus is provided for attaching a pair of sunglasses to headgear, and it includes at least one semi-rigid strip of a predetermined material having one side at least partially coated with an adhesive suitable for adhering to the top side of a bill on a visor, a cap, or a bicycle helmet; and, a pair of adhesive backed hook and loop pads trimmable to be suitably sized to adhere to lowest underside portion of the plastic frames of the sunglasses.

### OBJECTS OF THE INVENTION

It is, therefore, one of the primary objects of the present invention to provide a kit for readily attaching devices to virtually any hat or visor, and to most common sunglasses for the purpose of securely holding sunglasses when they are not being worn.

Another object of the present invention is to provide economical after-market attachments enabling rapid on and off relocation of sunglasses between the face and retention devices on the headgear without removal of the headgear.

An additional object of the present invention is to provide a versatile attachment means for sunglasses with either straight or curved temples, and which can be attached to existing ski hats and bicycle helmets as well as caps and visors.

In addition to the various objects and advantages of the present invention described with some degree of specificity above it should be obvious that additional objects and advantages of the present invention will become more readily apparent to those persons who are skilled in the relevant art from the following more detailed description of the invention, particularly, when such description is taken in conjunction with the attached drawing figures and with the appended claims.

### BRIEF DESCRIPTION OF THE DRAWINGS

FIG. 1 is an overview of a sunvisor with a pair of sunglasses held to it with devices of the present invention.

FIG. 2 shows a ball cap with one of the devices attached, but without the sunglasses in place.

FIG. 3 illustrates the attachment of hook and loop pads to the inside temples of a pair of sunglasses.

FIG. 4 is a top view of the assembly of parts into the two main pieces

FIG. 5 shows the devices in use on a ski hat prior to attachment to a hat.

FIG. 6 depicts the pieces packaged into a kit.

FIG. 7 shows an alternative embodiment deployed on a ball cap.

### BRIEF DESCRIPTION OF A PRESENTLY PREFERRED AND ALTERNATIVE EMBODIMENT OF THE INVENTION

Prior to proceeding to the more detailed description of the present invention it should be noted that, for the sake of clarity and understanding, identical components which have identical functions have been identified with identical reference numerals throughout the several views illustrated in the drawing figures.

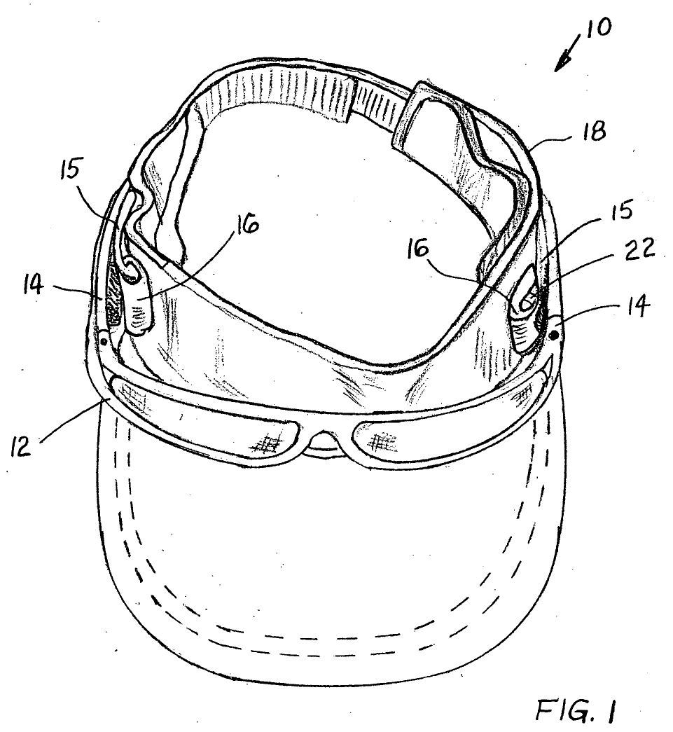

Referring to FIG. 1, an overview of a deployment of the kit on a visor/sunglasses combination 10 is shown. Sunglasses 12, with Velcro-like pads 14 adhered to the inside of the temples proximal the hinges, are held snugly to mating Velcro-like strips 15 looped at the front 16 and pinned to the sides of the visor 18 with a dual-prong pin 22 at the front.

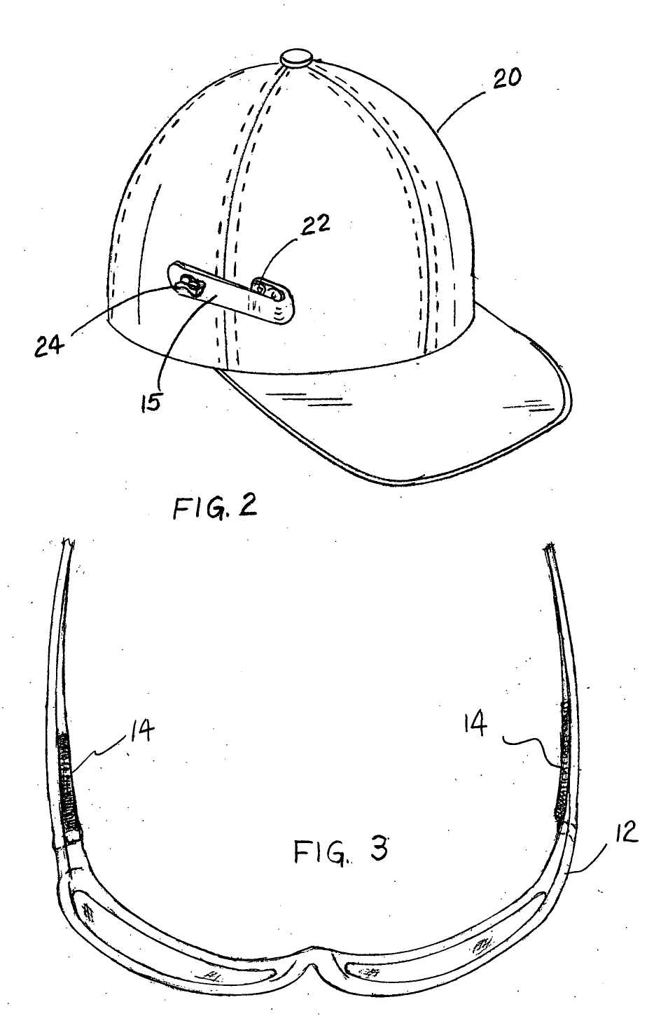

In FIG. 2, one of the two hook and loop strips 15 is shown looped and attached to a ball cap 20 by means of a dual-prong pin 22 and a single prong pin 24. A matching arrangement would be similarly disposed on the opposite side, and together they would grab and securely hold a pair of sunglasses equipped with mating hook and loop (Velcro) pads on the forward inside area of the temples. With only a single-prong pin near the front loop, the loop tends to rotate out of position when the glasses are removed, or not in place. The dual-prong pin prevents this from occurring. Alternatively, an adhesive section on the functional side of the Velcro strip could hold the strip to the cap.

In FIG. 3, the sunglasses 12 are shown with the mating Velcro pads 14 stuck to the inside of the temples starting at the hinges extending rearward for about three centimeters.

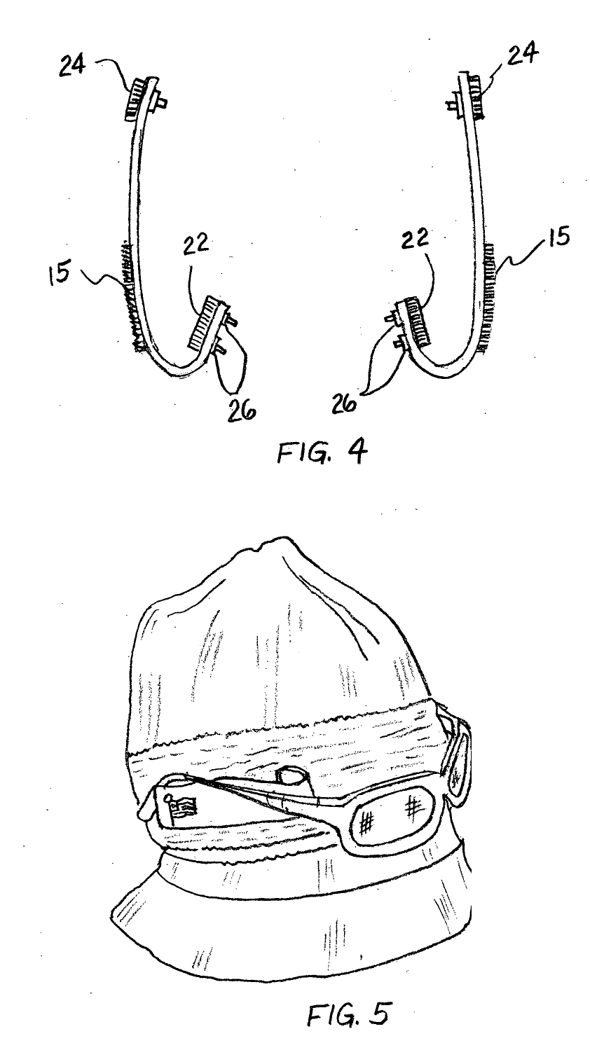

Referring to FIG. 4, the pair of Velcro strips 15 are shown nearly ready for installation on a hat, with the dual-prong pins 22 already attached at the U-bend end and the single-prong pins 24 at the distal end. The actual Velcro portion of the strips 15 needs to cover only the outer portion near the loop end. To install, the backing clasps 26 are removed and the dual-prong pins 22 are pushed through the cap material at the appropriate frontal/side locations, and the clasps 26 are reinstalled. Subsequently, the strips are extended rearward on the hat forming a U-bend covering the double-prong pins, and the single pins pierce through the visor or cap at a location about midway back on the sides.

FIG. 5 illustrates how the invention could also be used on a ski cap.

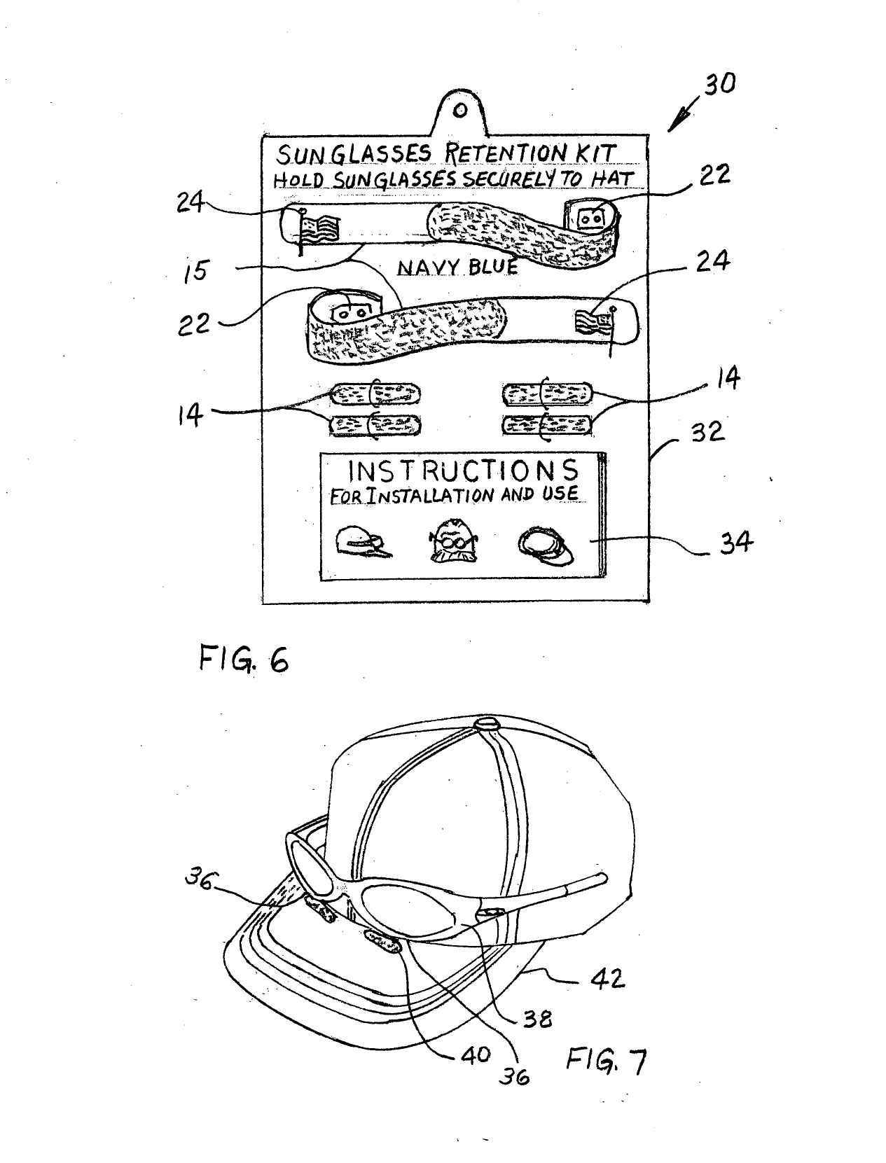

FIG. 6 depicts all the various parts assembled into a kit 30. The Velcro strips 15, offered in various colors, would be pinned to the cardboard backing 32 with the dual-prong pins 22 and the single-prong pins 24. Two sets of Velcro pads 14 would be included in two different colors. These pads would need to be the opposite functionality (hook or loop) as needed to mate to the strips 15. Also included would be an instruction pamphlet 34 with small photographs on the outside depicting various applications for the kit. Although not shown, double-sided tape may also be included to provide a means of attachment to helmets as well. Typically, at least the entire front side would be covered with semi-rigid molded clear plastic.

An alternative, but similar, embodiment of the invention, shown in FIG. 7 would be one in which the Velcro pads 36 are stuck to the underside of the lens frames 38, and these would mate up with one or two mating Velcro strips 40 adhered to the top surface of the bill 42 near the seam.

While a presently preferred and an alternative embodiment of the present invention have been described in sufficient detail above to enable a person skilled in the relevant art to make and use the same it should be obvious that various other adaptations and modifications can be envisioned by those persons skilled in such art without departing from either the spirit of the invention or the scope of the appended claims.

I claim:

1. A sunglasses-retention kit to enable attachment of sunglasses to preselected headgear, said kit comprising:
a) at least one substantially flexible hook and loop strip engageable to said preselected headgear and having each of a predetermined length and a predetermined width, wherein said hook and loop strip includes hooks/loops over at least one-third of said strip; and
b) at least one adhesive-backed hook and loop pad engageable to said sunglasses for mating to said flexible hook and loop strip, having each of a predetermined length and a predetermined height.

2. The sunglasses-retention kit, according to claim 1, wherein said attachment means includes:
a) frontal attachment means disposed on one end of said hook and loop strip for attaching to said preselected headgear; and
b) side attachment means disposed on said hook and loop strip at side and end opposite said frontal attachment means for attaching to said preselected headgear such as to create a U-shaped loop of outward-facing hook and loop material adjacent said frontal attachment means.

3. The sunglasses-retention kit, according to claim 1, wherein said kit further includes written instructions in at least one language describing how to install parts of said kit on one of a ball cap, golf hat, visor, helmet, and ski hat.

4. The sunglasses-retention kit, according to claim 1, wherein said predetermined length of said flexible hook and loop strips is between about six and twenty centimeters.

5. The sunglasses-retention kit, according to claim 1, wherein said predetermined height of said flexible hook and loop strip is between about one and four centimeters.

6. The sunglasses-retention kit, according to claim 2, wherein said frontal attachment means is a double-pronged pin with matching clasps, with prongs being between about five and twenty-five millimeters in length.

7. The sunglasses-retention kit, according to claim 2, wherein said side attachment means is a single-prong pin with matching clasp, said prong being between about five and eighteen millimeters in length.

8. The sunglasses-retention kit, according to claim 1, wherein said predetermined length of said adhesive-backed hook and loop pads is between about one centimeter and six centimeters.

9. The sunglasses-retention kit, according to claim 1, wherein said predetermined height of said flexible hook and loop pad is adjustable by trimming to between about three and twenty millimeters.

10. The sunglasses-retention kit, according to claim 6, wherein said double prong pin comes pre-attached to said flexible hook and loop strip with removable clasps in place.

11. The sunglasses-retention kit, according to claim 7, wherein said single prong pin comes pre-attached to said flexible hook and loop strip with removable clasp in place.

12. The sunglasses-retention kit, according to claim 2, wherein a substantial length near one end of each said flexible hook and loop strips is adhered to a substantially rigid U-shaped member which may also be penetrated by said dual-prong pins.

13. The sunglasses-retention kit, according to claim 1, wherein said flexible hook and loop strips are offered in one of various color choices per kit.

14. The sunglasses-retention kit, according to claim 1, wherein said adhesive-backed hook and loop pad is offered in at least one color per kit.

15. The sunglasses-retention kit, according to claim 7, wherein said single-prong pin is offered in at least one of various decorative choices per kit.

16. The sunglasses-retention kit, according to claim 7, wherein said single-prong pins are replaceable with an outward decorative design of owner's choice.

17. The sunglasses-retention kit, according to claim 1, wherein said attachment means for said hook and loop strip is at least one adhesive area on at least a portion of said hook and loop strip.

18. The sunglasses-retention kit, according to claim 2, wherein said frontal attachment means is an adhesive on an end and side containing said hooks and loops strips.

19. The sunglasses-retention kit, according to claim 2, wherein said frontal and side attachment means are double-sided tape.

20. An apparatus for attaching a pair of sunglasses to headgear, said apparatus comprising:
a) at least one semi-rigid strip of a predetermined material having each of a predetermined length, a predetermined width, wherein said semi-rigid strip has one side at least partially coated with an adhesive suitable for adhering to the top side of a bill on one of a cap and a visor; and
b) a pair of adhesive backed hook and loop pads trimmable to be suitably sized to adhere to lowest underside portion of plastic frames of said sunglasses.

21. The apparatus, according to claim 20, wherein said predetermine length of said semi-rigid strip is between about 15 and 25 centimeters.

22. The apparatus, according to claim 20, wherein said predetermined width of said semi-rigid strip is between about one and four centimeters.

23. The apparatus, according to claim 20, wherein said predetermined material is a hook and loop fabric.

24. The apparatus, according to claim 20, wherein said predetermined material is flexible vinyl tubing slit longitudinally along topside.
